(19) United States
(12) Patent Application Publication
(10) Pub. No.: US 2023/0158265 A1
(43) Pub. Date: May 25, 2023

(54) BREATHING ASSISTANCE APPARATUS

(71) Applicant: Gregory Calderiso, Lincoln, RI (US)

(72) Inventor: Gregory Calderiso, Lincoln, RI (US)

(21) Appl. No.: 17/809,325

(22) Filed: Jun. 28, 2022

Related U.S. Application Data
(60) Provisional application No. 63/264,516, filed on Nov. 24, 2021.

(51) Int. Cl. A61M 16/04; A61M 16/00

(52) U.S. Cl. CPC A61M 16/0493; A61M 16/0066; A61M 2205/35; A61M 2205/502; A61M 2205/582; A61M 2205/8206; A61M 2205/8262

(57) ABSTRACT

A breathing apparatus includes a mouthguard that fits on a user’s upper teeth, a breathing channel with an airway protruding from a central portion of the mouthguard, and a motor housed within the breathing apparatus operated by a circuit board.

### CROSS-REFERENCE TO RELATED APPLICATION

This application claims the benefit of priority of U.S. Provisional Application No. 63/264,516, filed Nov. 24, 2021, the contents of which are herein incorporated by reference.

### BACKGROUND OF THE INVENTION

The present invention relates to a breathing apparatus and, more particularly, to a breathing assistance apparatus.

During the course of the day, a person’s breathing may become shallow, and their respiration rate is thrown out of an ideal rhythm. This leads to unnecessary decreases in heart rate variability (HRV) and additionally leads to stress and anxiety by means of a sympathetic, flight or fight, reaction in the body. Ideally, the body is in a parasympathetic and calm state. This ideal state may be achieved through proper breathing techniques.

Currently, breathing techniques are employed to assist a person in controlling their respiration rate and rhythm. However, these techniques rely on a person’s focus and concentration. In addition, a person must be taught the techniques. A forgetful person will have trouble implementing these commonly employed breathing techniques. More commonly, it’s difficult to stay focused on these breathing techniques. A person with a short attention span will often find their minds wandering when attempting to exercise breathing controls, resulting in no gains from the exercise.

As can be seen, there is a need for a device that guides a user in implementing breathing techniques and keeps their attention focused on their breathing.

### SUMMARY OF THE INVENTION

In one aspect of the present invention, a breathing apparatus comprises a mouthguard operative to fitting on a user’s upper teeth, a breathing channel with an airway protruding from a central portion of the mouthguard, and a motor housed within the breathing apparatus operated by a circuit board.

In another aspect of the present invention, a breathing apparatus comprises a mouthguard, a breathing channel underneath a middle portion of the mouthguard, and a chamber within the breathing apparatus housing a fan, a motor, and a circuit board wherein the fan provides a suction through the breathing channel.

In another aspect of the present invention, a breathing apparatus comprises a mouthguard, a breathing channel underneath a middle portion of the mouthguard, and a chamber within the breathing apparatus housing a circuit board and a vibrational motor.

These and other features, aspects and advantages of the present invention will become better understood with reference to the following drawings, description, and claims.

### BRIEF DESCRIPTION OF THE DRAWINGS

FIG. 1 is a top perspective view of a breathing apparatus according to an embodiment of the present invention;

FIG. 2 is a side elevation view thereof in use;

FIG. 3 is a front elevation view thereof;

FIG. 4 is a schematic sectional view thereof; and

FIG. 5 is a top perspective of a breathing apparatus view according to an embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

A general overview of the various features of the invention will be provided, with a detailed description following. Broadly, an embodiment of the present invention provides a breathing apparatus. The breathing apparatus may assist a user in controlling their breathing and implementing breathing techniques. The apparatus may guide a user in controlling their breathing by prompting inhales or exhales. The prompts may cue or signal the user, for example, by a fan or a vibration. The fan may provide a suction or forcing air into a user’s mouth. The apparatus signals the user according to a breathing rhythm or pattern which may be selected by the user.

The apparatus may be placed in a user’s mouth. A breathing pattern may be selected by the user from a selection of pre-selected or installed patterns. The breathing patterns may be selected via an app in electronic communication with the apparatus or by at least one button on an exterior of the apparatus. The app may have a feature enabling customization of a breathing pattern, such as a personalized resonance breathing frequency. Multiple pattern options may be included in one apparatus. A selection device on an outside of the apparatus may enable a user to select a pattern. The patterns may correspond to patterns of suction of a fan within the apparatus or patterns of vibration of a motor within the apparatus. Said patterns of suction or patterns of vibration may be an inhale and/or exhale cue and may signal the user at specified times according to a selected pattern. The signal prompts the user to breathe in or breathe out. The signal may mimic breathing sensations through air suction in and/or out of the user’s mouth.

A mouth guard may fit into the mouth of the user on the user’s upper teeth. A protuberance with a tunnel may be molded into a center portion of the apparatus and extend into the user’s mouth, between the user’s lips. A user’s tongue may rest underneath the tunnel while the user is breathing.

A chamber is housed within the breathing apparatus. The chamber may comprise fans, a motor, a circuit board, an antenna, a switch, and a USB charging port. The faceplate may be used for the placement of a trademark or product details. The location of the chamber within the apparatus or the shape of the chamber is not particularly limited. The chamber may be covered by a faceplate.

A button on an exterior of the apparatus may be pressed to begin a session. A timer may control the length of the session. Examples of the length of a session include 5 minutes, 10 minutes, 15 minutes, and 20 minutes.

In some embodiments of the present invention, when the apparatus is powered on, fans may create a suction to mimic an inhale and/or exhale of the user. The suction may last for a set time depending on the selected breathing pattern. The suction encourages and enables the user to mimic the pattern of the suction, thus assisting the user in controlling their respiration rate. The user’s respiration rate may be controlled with minimal effort while maintaining focus on other tasks.

A motor may drive the fans. The fans may apply the suction to the tunnel. The motor is attached to a circuit board controlling a flow and timing of the fans. A battery may power the motor. The battery may be rechargeable via a USB port.

In some embodiments of the invention the motor may provide vibrations. In said embodiments, a vibrational motor may be used. Said embodiments may not include fans. The motor may vibrate according to the desired breathing pattern. The vibrations cue the user to inhale or exhale in accordance with the desired breathing pattern.

The apparatus may communicate via the antenna with an app on a computer or mobile device. A user may input information into the app such as gender, age, occupation, activity levels, perceived stress levels, perceived sleep quality, and other information relevant to a user’s breathing. The app may send prompts or push notifications to the user. Baselines may be generated from continuous user feedback and data collected by the apparatus. Said baselines may be recorded by the app. These baselines may be used to generate individualized breathing protocols and doses, time of sessions, for each user. The baselines and other data collected by the app may be stored in a central database that communicates with remote servers.

The user may select a breathing protocol and dose length through the app and initiate sessions through the app. Sessions initiated through the app may prompt for user input. The app may use machine learning algorithms to send automated breath dosing prompts that reflect how and when the user may benefit from the apparatus.

The application may include a virtual personal breathing assistant or coach. The user may interact with the coach through a keyboard via chat. The coach may recommend breathing protocols and doses.

The user may complete a survey to fine tune the coach with machine learning algorithms. Machine learning algorithms may also use collected data and baselines stored in the central database along with discussions with the coach to enhance the chat features and user experience.

The antenna may communicate back with a mobile device to transfer data about each dose/usage. This data may be stored in a database on a cloud platform. This data may be utilized train or develop the virtual breathing coach/assistant. The breathing coach/assistant may be part of the app and communicate prescribed doses to the user based on their past data, real-time input, and requests for a breathing dose.

In some embodiments, the present invention may allow for custom breathing patterns to be generated, entered, stored, and/or communicated from the mobile application to the antenna.

In some embodiments, the device may function as a complementary and integrative health approach for pain management, stress, depression, and anxiety treatment. It may enable remote patient monitoring such as through the app and data stored in the cloud or remote database.

In some embodiments of the present invention, the device activates, regulates, or manages a user’s parasympathetic nervous system. For example, carbon dioxide (CO2) may accumulate when a user holds their breath. Increasing blood CO2 may enhance a cardio-inhibitory response of a vagus nerve when exhaling and stimulate the parasympathetic nervous system. Some embodiments of the present invention may encourage a user accumulate CO2, producing a calm and relaxed feeling by the method or reaction described.

In some embodiments of the present invention, the device disallows a jaw to clench. Jaw clenching may trigger tension headaches, teeth damage, and/or disorder in the temporomandibular joint.

In addition, the device may reduce stress levels and reduce cortisol levels. The invention may be utilized when stressed. It may further facilitate the user’s natural relaxation response and help with asthma and sleep.

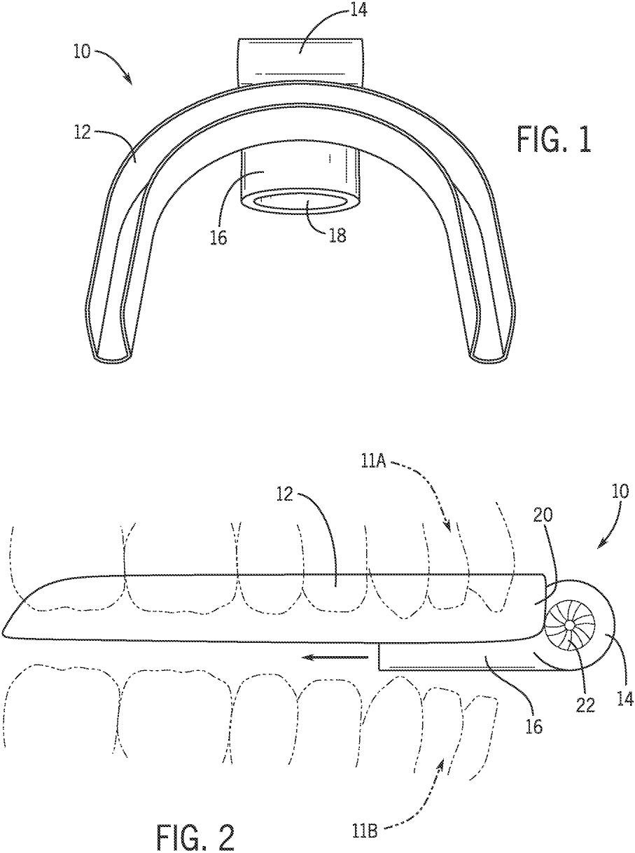

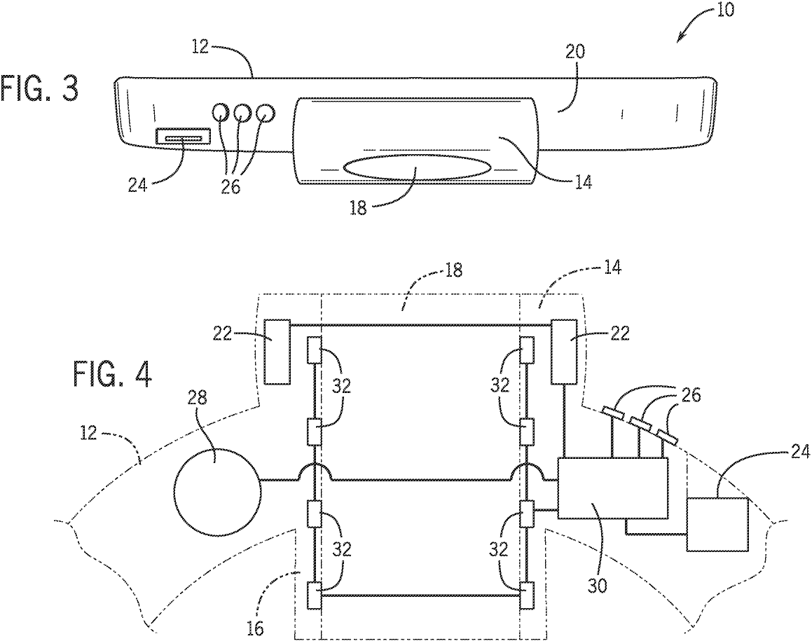

Referring now to the Figures, FIG. 1 shows a top perspective view of the apparatus 10 according to an embodiment of the present invention. A mouthguard 12 fits on a user’s upper teeth 11A (best seen in FIG. 2). A breathing channel 16 protrudes from a central, middle portion of the mouthguard 12. The breathing channel comprises an airway 18 with a first end and a second end for a user to breathe through. The first end of the airway 18 is best shown in FIG. 1 with the second end being best shown in FIG. 3. A chamber 14 is attached to an end of the breathing channel 16.

FIG. 2 shows the apparatus 10 in use according to an embodiment of the present invention. Upper teeth 11A fit into the mouthguard 12. The breathing channel 16 fits into the user’s mouth. Fans 22 are housed inside of the chamber 14. A faceplate 20 is on a front end of the mouthguard 12. The user’s lower teeth 11B may grip the mouthguard 12. The fans 22 provide suction through the airway 18.

FIG. 3 is a front view according to an embodiment of the present invention. Buttons 26 and a USB charging port 24 are on the front end of the mouthguard 12. In some embodiments, the present invention does not include the second end of the airway 18.

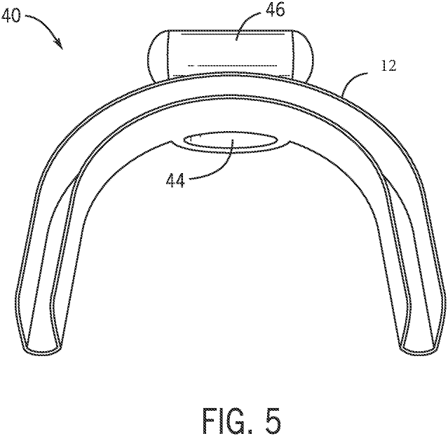

FIG. 4 is a diagram view according to an embodiment of the present invention. The buttons 26 and the USB charging port 24 are connected to a circuit board 30. The circuit board 30 is connected to the fans 22, motors 32, and a battery 28. The circuit board 30 may also connect to an antenna, such as Bluetooth™ antenna (not pictured).

FIG. 5 shows an alternate embodiment of the present invention 40. An airflow opening 44 is attached to a mouthguard 12. A chamber 46 of said includes a vibrational device such as a vibrational motor.

It should be understood, of course, that the foregoing relates to exemplary embodiments of the invention and that modifications may be made without departing from the spirit and scope of the invention as set forth in the following claims.

What is claimed is:

1. A breathing apparatus comprising:
a mouthguard operative to fitting on a user’s upper teeth;
a breathing channel with an airway protruding from a central portion of the mouthguard; and
a motor housed within the apparatus operated by a circuit board.

2. The breathing apparatus of claim 1, further comprising an antenna, operative to communicating a selection by a user on an app of a pattern and a dose to the circuit board.

3. The breathing apparatus of claim 1, further comprising buttons on an exterior of the breathing apparatus operative to communicating a selection by the user of a pattern and dose to the circuit board.

4. The breathing apparatus of claim 1, further comprising a battery rechargeable by a USB port on an exterior of the breathing apparatus.

5. The breathing apparatus of claim 1, further comprising fans operative to providing a suction through the breathing channel.

6. The breathing apparatus of claim 5, wherein the motor is operative to driving said fans.

7. The breathing apparatus of claim 6, wherein the motor electronically communicates with the circuit board to provide suction from the fans in a pattern selected by the user.

8. The breathing apparatus of claim 1, wherein the motor is a vibrational motor.

9. The breathing apparatus of claim 8, wherein the motor electronically communicates with the circuit board to provide vibrations in a pattern selected by the user.

10. A breathing apparatus comprising:
a mouthguard;
a breathing channel underneath a middle portion of the mouthguard; and
a chamber within the breathing apparatus housing a fan, a motor, and a circuit board wherein the fan provides a suction through the breathing channel.

11. The breathing apparatus of claim 10, further comprising at least one button on an exterior of the breathing apparatus, wherein the at least one button is in electronic communication with the fan through the circuit board and operative to selecting a pattern of suction for the fan.

12. A breathing apparatus comprising:
a mouthguard;
a breathing channel underneath a middle portion of the mouthguard; and
a chamber within the breathing apparatus housing a circuit board and a vibrational motor.

13. The breathing apparatus of claim 12, further comprising at least one button on an exterior of the breathing apparatus, wherein the at least one button is in electronic communication with the vibrational motor through the circuit board and operative to selecting a pattern of vibration for the motor.
